(19) United States
(12) Patent Application Publication
(10) Pub. No.: US 2026/0011430 A1
(43) Pub. Date: Jan. 8, 2026

(54) DEVICE FOR RECOMMENDING PATIENT-CUSTOMIZED TRAINING CONTENT AND METHOD THEREOF

(71) Applicant: MINDHUB INC., Anyang-si (KR)

(72) Inventor: Hae Sung LEE, Anyang-si (KR)

(73) Assignee: MINDHUB INC., Anyang-si (KR)

(21) Appl. No.: 19/259,039

(22) Filed: Jul. 3, 2025

Related U.S. Application Data
(63) Continuation of application No. PCT/KR2023/021642, filed on Dec. 27, 2023.

(30) Foreign Application Priority Data
Jan. 6, 2023 (KR) 10-2023-0002222

(51) Int. Cl. G16H 20/70

(52) U.S. Cl. CPC G16H 20/70

(57) ABSTRACT

Disclosed are a patient-customized training content recommendation device and a method thereof. The device includes a profile receiver that receives a patient profile including basic information and cognitive information of a patient, a cognitive ability evaluating unit that calculates a function-specific cognitive score of the patient by evaluating a cognitive ability of the patient based on the patient profile, a training recommendation unit that recommends at least one training content through an artificial intelligence (AI) rehabilitation recommendation model, and a training conducting unit that calculates a result for the training content provided to the patient through a user terminal.

### CROSS-REFERENCE TO RELATED APPLICATIONS

The present application is a continuation of International Patent Application No. PCT/KR2023/021642, filed on Dec. 27, 2023, which is based upon and claims the benefit of priority to Korean Patent Application No. 10-2023-0002222 filed on Jan. 6, 2023. The disclosures of the above-listed applications are hereby incorporated by reference herein in their entirety.

### BACKGROUND

Embodiments of the present disclosure described herein relate to a training content recommendation device and a method thereof, and more particularly, relate to a patient-customized training content recommendation device based on a label code, and a method thereof.

Cognitive disorders are becoming major social problems as modern societies enter an aging population. Because there are no specially developed drugs, it is difficult to treat these cognitive disorders.

Accordingly, it is important to diagnose and prevent cognitive disorders at an early stage and slow down the progression of cognitive disorders, and there is a growing consumer demand for training content to treat patients.

### SUMMARY

Embodiments of the present disclosure provide training content suited to a patient by identifying the patient's cognitive deficiencies.

Embodiments of the present disclosure provide a predicted score by predicting a score for a cognitive ability evaluation method different from a training method of obtaining the corresponding cognitive score by calculating a cognitive score.

Problems to be solved by the present disclosure are not limited to the problems mentioned above, and other problems not mentioned will be apparent by those skilled in the art from the following description.

According to an embodiment, a patient-customized training content recommendation device includes a profile receiver that receives a patient profile including basic information and cognitive information of a patient, a cognitive ability evaluating unit that calculates a function-specific cognitive score of the patient by evaluating a cognitive ability of the patient based on the patient profile, a training recommendation unit that recommends at least one training content through an artificial intelligence (AI) rehabilitation recommendation model, and a training conducting unit that calculates a result for the training content provided to the patient through a user terminal.

According to an embodiment, a patient-customized training content recommending method includes receiving a patient profile including basic information and cognitive information of a patient, calculating a function-specific cognitive score of the patient by evaluating a cognitive ability of the patient based on a patient profile, recommending at least one training content through an AI rehabilitation recommendation model, and calculating a result for the training content provided to the patient through the user terminal.

Besides, a computer program stored in a computer-readable recording medium for executing a method to implement the present disclosure may be further provided.

In addition, a computer-readable recording medium for recording a computer program for performing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a drawing illustrating a patient-customized training content recommendation system, according to an embodiment of the present disclosure;

FIG. 2 is a drawing illustrating a physical configuration of a patient-customized training content recommendation device, according to an embodiment of the present disclosure;

FIG. 3 is a diagram illustrating a functional configuration of a patient-customized training content recommendation device, according to an embodiment of the present disclosure;

FIG. 4 is a diagram illustrating an order in which a patient-customized training content recommending method according to an embodiment of the present disclosure is performed;

FIG. 5 is a drawing for describing a main screen in which a patient-customized training content recommendation device according to an embodiment of the present disclosure is implemented;

FIG. 6 is a drawing illustrating a screen providing a patient list through a patient-customized training content recommendation device, according to an embodiment of the present disclosure;

FIGS. 7A and 7B are diagrams for describing training content provided by a patient-customized training content recommendation device, according to an embodiment of the present disclosure;

FIGS. 8A and 8B are diagrams for describing training content recommended by a patient-customized training content recommendation device, according to an embodiment of the present disclosure; and

FIG. 9 is a diagram for describing the result of training content provided by a patient-customized training content recommendation device, according to an embodiment of the present disclosure.

### DETAILED DESCRIPTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

In this specification, a ‘device according to an embodiment of the present disclosure’ includes all various devices capable of providing results to a user by performing arithmetic processing. For example, the apparatus according to an embodiment of the present disclosure may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

The server device may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet (WiBro) terminal, and a wearable device such as a timepiece, a ring, a bracelet, an anklet, a necklace, glasses, a contact lens, or a head-mounted device (HMD).

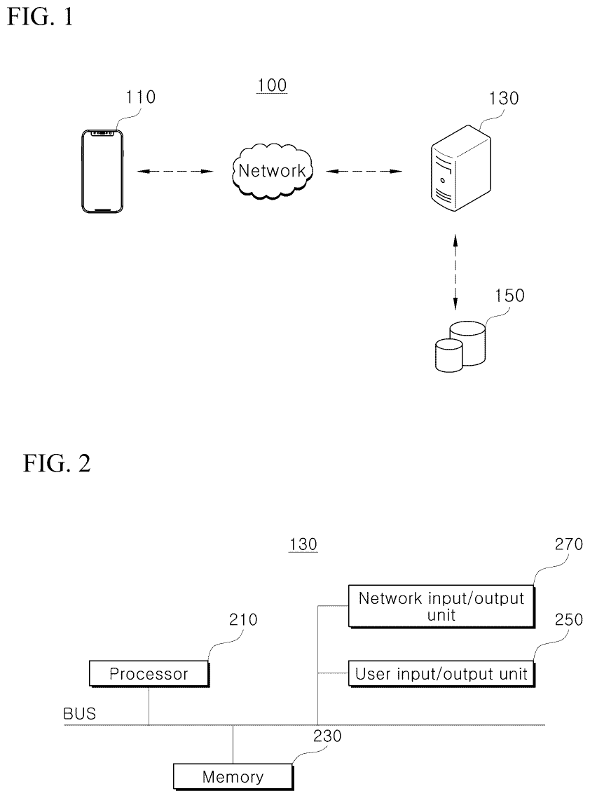

FIG. 1 is a drawing illustrating a patient-customized training content recommendation system 100, according to an embodiment of the present disclosure.

Referring to FIG. 1, the system 100 that recommends patient-customized training content may include a user terminal 110, a device 130 that recommends patient-customized training content, and a database 150.

The user terminal 110 may identify a patient profile, which is collected through the patient-customized training content recommendation device 130, a function-specific cognitive score, and training content, which is provided through the patient-customized training content recommendation device 130. In addition, the user terminal 110 may be implemented as a smartphone or a wearable device that may provide the patient profile to the patient-customized training content recommendation device 130, and is not necessarily limited thereto. The user terminal 110 may also be implemented as various devices such as tablet PCs. The user terminal 110 may be connected to the patient-customized training content recommendation device 130 via a network. The plurality of user terminals 110 may be simultaneously connected to the patient-customized training content recommendation device 130.

The patient-customized training content recommendation device 130 may be implemented as a server corresponding to a computer or a program that sequentially performs operations of receiving the patient profile, calculating the function-specific cognitive score of a patient by evaluating the patient's cognitive ability based on the patient profile, recommending the training content based on the acquired function-specific cognitive score, and receiving performance information about the provided training content. The patient-customized training content recommendation device 130 may be wirelessly connected to the user terminal 110 via Bluetooth, WiFi, a communication network, etc., and may exchange data with the user terminal 110 via a network.

The database 150 may correspond to a storage device that stores various pieces of information generated through operations of receiving the patient profile, calculating the function-specific cognitive score of a patient by evaluating the patient's cognitive ability based on the patient profile, recommending the training content based on the acquired function-specific cognitive score, and receiving performance information about the provided training content.

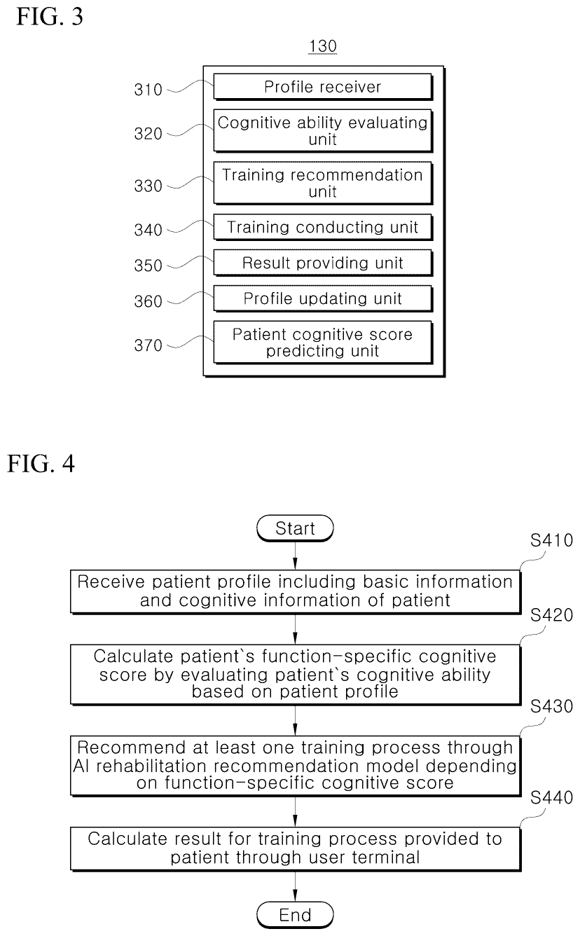

FIG. 2 is a drawing illustrating a physical configuration of the patient-customized training content recommendation device 130, according to an embodiment of the present disclosure.

Referring to FIG. 2, the patient-customized training content recommendation device 130 may be implemented to include a processor 210, a memory 230, a user input/output unit 250, and a network input/output unit 270.

The processor 210 may perform a procedure of operations of receiving a patient profile, calculating a function-specific cognitive score of a patient by evaluating the patient's cognitive ability based on the patient profile, recommending training content based on the obtained function-specific cognitive score, and receiving performance information about the provided training content, may manage read and write operations of the memory 230 throughout the procedure, and may schedule synchronization timing between a volatile memory and a non-volatile memory in the memory 230. The processor 210 may control the overall operation of the patient-customized training content recommendation device 130, and may be electrically connected to the memory 230, the user input/output unit 250, and the network input/output unit 270 to control the data flow therebetween. The processor 210 may be implemented as a central processing unit (CPU) of the patient-customized training content recommendation device 130.

The memory 230 may include an auxiliary memory device, which is implemented with a non-volatile memory, such as a Solid State Drive (SSD) or a Hard Disk Drive (HDD) and which is used to store all data required for the patient-customized training content recommendation device 130, and may include a main memory device implemented with a volatile memory such as a Random Access Memory (RAM).

The user input/output unit 250 may include an environment for receiving a user input and an environment for outputting specific information to a user. For example, the user input/output unit 250 may include an input device including an adapter such as a touch pad, a touch screen, a virtual keyboard, or a pointing device, and an output device including an adapter such as a monitor or a touch screen. In the present disclosure, the user input/output unit 250 may correspond to a computing device accessed via remote access. In this case, the patient-customized training content recommendation device 130 may operate as a server.

The network input/output unit 270 includes an environment for connecting to an external device or system via a network, and may include adapters for communication such as Local Area Network (LAN), Metropolitan Area Network (MAN), Wide Area Network (WAN), and Value Added Network (VAN).

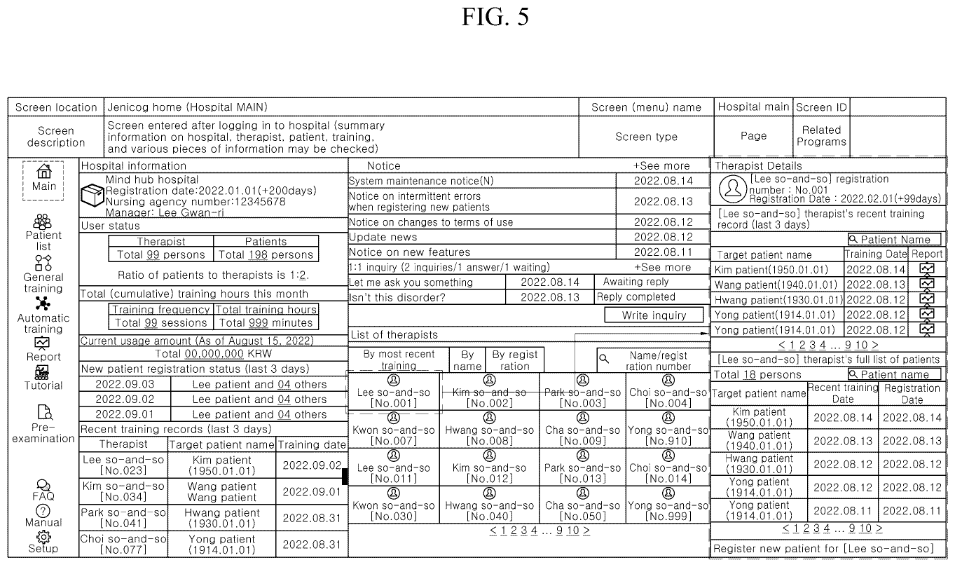

FIG. 3 is a diagram illustrating a functional configuration of the patient-customized training content recommendation device 130, according to an embodiment of the present disclosure. Referring to FIG. 3, the patient-customized training content recommendation device 130 may include a profile receiver 310, a cognitive ability evaluating unit 320, a training recommendation unit 330, a training conducting unit 340, a result providing unit 350, a profile updating unit 360, and a patient cognitive score predicting unit 370.

The profile receiver 310 may receive a patient profile including basic information and cognitive information of a patient. The patient's basic information may include the patient's age, gender, highest level of education, and current status. In this case, the current status may include at least one of cognitive and language severity of the patient. The cognitive information may include the result for training content, and may include a separately pre-defined cognitive score when there is no training content already performed by the patient. Here, the already-performed training content may mean training content performed through the patient-customized training content recommendation device 130. That is, when there is training content already performed by the patient, the profile receiver 310 may receive the patient's cognitive score according to the result of the training content.

Here, the predefined cognitive score may mean a separate cognitive score, which is different from the result of the training content performed through the patient-customized training content recommendation device 130. For example, the predefined cognitive score may refer to a score associated with measuring cognitive ability, such as Mini-mental State Examination (MMSE), Global Deterioration Scale (GDS), and Clinical Dementia Rating (CDR).

The cognitive ability evaluating unit 320 may calculate the patient's function-specific cognitive score by evaluating the patient's cognitive ability based on the patient profile. For example, the cognitive ability evaluating unit 320 may calculate a cognitive score for each of the patient's cognitive functions, including attention, memory, executive function, reading, writing, speaking, and comprehension. In more detail, the cognitive ability evaluating unit 320 may calculate absolute scores and statistical scores for the patient's cognitive function, which are described in detail below.

In the present disclosure, the cognitive ability evaluating unit 320 may define a function-specific cognitive score depending on sub-functions split stepwise, and may calculate a statistical value for each sub-function. For example, the sub-functions thus split stepwise may be broadly categorized as mental functions. The mental functions may include perceptual, executive, language, computational, and memory functions. Moreover, the perceptual function may include auditory perception, visual perception, and spatiotemporal perception. Furthermore, the executive function may include abstraction, organization and planning, cognitive flexibility, determination, and problem solving. The language function may include understanding of language and expressing of language. The computational function may include simple and complex calculations. In this way, the sub-functions may be split into major categories, medium categories, and minor categories, but are not limited thereto. Accordingly, the cognitive ability evaluating unit 320 may define sub-functions in the same way as described below by defining at least one major categories and placing medium categories under the major categories.

Besides, the cognitive ability evaluating unit 320 may calculate a statistical value for each sub-function. For example, the cognitive ability evaluating unit 320 may calculate the statistical value for each sub-function in such a way that the score for a mental function is in the top 10% of patients of the same age. Here, the statistical value may mean statistically processed data, such as calculating not only percentiles but also statistically processed z-scores, and a difference from same-age groups to a calculate p-value.

In the present disclosure, the cognitive ability evaluating unit 320 may define a function-specific cognitive score through a first step of defining at least one sub-function, a second step of defining a detailed sub-function included in at least one sub-function, and a third step of performing labeling on the sub-function and the detailed sub-function to generate a label code for the sub-function and the detailed sub-function. For example, the cognitive ability evaluating unit 320 may define sub-functions as a mental function, a sensory function and pain, and a voice and speech function. Moreover, the cognitive ability evaluating unit 320 may define that a sensory function and a pain function include detailed sub-functions of a visual function, a function of the structure around eyes, and an auditory function.

Furthermore, the cognitive ability evaluating unit 320 may assign a label code of b1 to the mental function, may assign a label code of b2 to the sensory function and pain, and may assign a label code of b3 to the voice and speech function. Besides, the cognitive ability evaluating unit 320 may assign label codes to detailed sub-functions, and, for example, may assign label code b210 to the visual function.

In the present disclosure, the cognitive ability evaluating unit 320 may calculate a function-specific cognitive score by summing statistical values for each sub-function. For example, the cognitive ability evaluating unit 320 may calculate statistical values for sub-functions of a voice function, an articulation function, a function of speaking, fluency, and rhythm, and an alternative vocalization function to add statistical values, and may calculate a cognitive score for a voice and speech function corresponding to the sub-function.

The training recommendation unit 330 may recommend at least one training content through an artificial intelligence (AI) rehabilitation recommendation model based on a function-specific cognitive score. For example, the training recommendation unit 330 may recommend training content for training a function lower than a typical cognitive function of a person having the same age as the patient's age among the patient's function-specific cognitive scores. For example, the training recommendation unit 330 may recommend training content capable of training attention for a patient with attention deficit disorder, as described in more detail below.

The training content may include at least one cognitive training with the set training type, difficulty level, number of problems, and solution time. For example, the training recommendation unit 330 may provide training content to the patient while setting the type of training, the difficulty, the number of problems, and the solution time.

In the present disclosure, the training recommendation unit 330 may provide training content for reinforcing a sub-function with a low statistical value. For example, when the statistical value of the mental function of the corresponding patient is low, the training recommendation unit 330 may provide training content for supplementing the corresponding mental function.

In the present disclosure, the training recommendation unit 330 may provide training content for reinforcing a detailed sub-function with a low statistical value. For example, the training recommendation unit 330 may provide training content for improving memory to a patient with a low statistical value for memory.

In the present disclosure, the training recommendation unit 330 may determine to limit a criterion with a low statistical value to a certain number. For example, the training recommendation unit 330 may provide training content for reinforcing four lower sub-functions with low scores among the sub-functions. For another example, the training recommendation unit 330 may provide training content for protecting sub-functions having a “z-score of −1.5” or lower.

In the present disclosure, the training recommendation unit 330 may recommend at least one training content by selecting cognitive training including the most label codes of detailed sub-functions included in sub-functions with low statistical values through the AI rehabilitation recommendation model. For example, when an executive function label code among the detailed sub-functions is b164, and training codes for associating a word include b1640, b1641, b1642, and b1643, the word association training may include four training codes belonging to the subset of b164, and thus the training recommendation unit 330 may provide the corresponding training to the patient by including the corresponding training in the training content. That is, the training recommendation unit 330 may identify a label code of the training included in the corresponding training, and may determine whether to recommend the corresponding training while the corresponding training is included in the training content, by reviewing whether the patient matches an insufficient label code.

In the present disclosure, the training recommendation unit 330 may recommend a recommended training content list as training content. Here, the AI rehabilitation recommendation model may generate the recommended training content list including a specific label code depending on the input of the specific label code. For example, the training recommendation unit 330 may receive a specific label code that requires training for a patient who has visited for the first time, and may provide training content corresponding to the corresponding label code through the AI rehabilitation recommendation model.

In the present disclosure, the AI rehabilitation recommendation model may generate the recommended training content list based on a cluster, to which the patient belongs, by clustering at least one patient based on the performance result for specific training content.

In detail, the AI rehabilitation recommendation model may collect accuracy and reaction time from the patient's performance result for the training content and may calculate the similarity in accuracy and reaction time with another patient. In other words, the AI rehabilitation recommendation model may determine at least one reference value, may calculate an n-dimensional numerical value including the corresponding reference value for each patient, and may calculate the similarity of each dimension and the similarity between specific patients by comparing the numerical values of each dimension. In an embodiment, the AI rehabilitation recommendation model may generate a patient cluster by calculating the cosine similarity between numerical values.

In the present disclosure, in addition to the training content performed by the patient, the AI rehabilitation recommendation model may generate, as the recommended training content list, training content performed by other patients in a cluster to which the patient belongs. For example, the AI rehabilitation recommendation model may generate, as the recommended training content list, training content, which is not performed by the corresponding patient in a cluster, to which the corresponding patient belongs, but is performed by other patients. For another example, the AI rehabilitation recommendation model may generate, as the recommended training content list, training content, which were most frequently performed by other patients, from among training content, which is not performed by the corresponding patient in the cluster to which the corresponding patient belongs, but is performed by other patients.

In the present disclosure, the AI rehabilitation recommendation model may generate the recommended training content list through a first step of determining whether a correct answer rate of the training content performed by a patient is greater than or equal to a first reference, a second step of determining whether a reaction time for training content performed by the patient is less than a second reference, when the correct answer rate of the patient is greater than or equal to the first reference, and a third step of generating the training content with increased difficulty as a recommended training content list when the difficulty of the training content performed by the patient is not the maximum, when the reaction time of the patient is less than the second reference. For example, the AI rehabilitation recommendation model may determine whether a reaction time for the training content performed by a patient is less than a certain time, when the correct answer rate for the training content performed by the patient is greater than or equal to 80%. Here, the specific time may be preset, and a value obtained by subtracting “2×median deviation” from a median response rate of patients in a cluster to which the patient belongs may be the specific time. When the patient's reaction time is less than the specific time, the AI rehabilitation recommendation model may increase the difficulty of the recommended training content by 1 when the difficulty of the training content performed by the patient is not the maximum, and may terminate the recommendation when the difficulty is the maximum. Moreover, the AI rehabilitation recommendation model may recommend training content for maintaining difficulty when the patient's reaction time is greater than or equal to the specific time.

In the present disclosure, the AI rehabilitation recommendation model may generate the recommended training content list through a first step of determining whether a correct answer rate of training content performed by a patient is less than a first reference, a second step of determining whether the correct answer rate of the training content performed by the patient is greater than or equal to a third reference, when the correct answer rate of the patient is less than the first reference, and a third step of generating training content having the previous difficulty as the recommended training content list when the correct answer rate of the patient is greater than or equal to the third reference, and generating training content having the reduced difficulty as the recommended training content list when the correct answer rate of the patient is less than the third reference. For example, the AI rehabilitation recommendation model may set a second reference as a value obtained by subtract “2×standard deviation” from the average correct answer rate according to the performance of the corresponding content by other patients in a cluster to which the corresponding patient belongs. Furthermore, the AI rehabilitation recommendation model may maintain or lower the difficulty of the recommended training content based on the correct answer rate of the corresponding patient.

In the present disclosure, the AI rehabilitation recommendation model may include a prediction model that predicts the result of a patient's training content, and a recommendation model that generates a recommended training content list based on a recommendation score calculated depending on a set target value. For example, the prediction model may predict the result of the patient's training content by using a learning method such as machine learning. In more detail, the prediction model may use a known AI learning method, and may predict the correct answer rate for the corresponding patient's training content through the results of the training performed by the patient. The recommendation model may generate the recommended training content list depending on the recommendation score calculated based on the set target value. For example, the recommendation model may calculate a recommendation score of training content based on target values for difficulty, performance experience, reaction time, solution time, and sensitivity. In more detail, the recommendation model may calculate the recommendation score according to Equations 1 to 11 below.

recommendation_score(p,t)=1+w1*difficulty(p,t,s1,s2)+w2*performance experience(p,t,s2)+w3*reaction_time(p,t,s3)+w4*solution_time(p,t,s4)+w5*sensitivity (p,t,s5,[ts]) [Equation 1]

(Here, w1 to w5 are predetermined weights; ‘p’ is a given patient, ‘t’ is the type of training, s1 to s5 are predetermined values; and [ts] is information about the completed training.)

difficulty(p,t,s1,s2)=w11*step(t,s1)+w12*timer(t,s1)+w13*correct_answer_rate(t,s1) [Equation 2]

step(t,s1)=1−|target(s1)−prediction(t)| [Equation 3]

(Here, target(s1) is s1/100; s1 is a target performance setting value; and prediction (t) is 1−(step (t)/maximum_step(t)).

timer(t,s1)=1−|target(s1)−prediction(t)| [Equation 4]

(Here, prediction (t) is idx(sort(timer_option_list(t)/len(timer_option_list(t)).)

correct answer rate(p,t,s1,s2)=w131*(1−|target(s1)−prediction(t)|) [Equation 5]

(Here, s2 is a performance experience preference setting value; w131 is a predetermined weight; and prediction (t) is a patient's predicted correct answer rate for the corresponding training content.)

Performance_experience(p,t,s2)=1−|target(s2)−prediction(p,t)| [Equation 6]

(Here, target(s2) is s2/100; s2 is a performance experience preference setting value; prediction (p, t) is 1/rank (t); and rank (t) is the location of ‘t’ in the sorted list sorted by the patient's history of performing the corresponding training content ‘t’ among entire training content ‘T’)

For example, when the patient has done a lot of that training content, the value of rank (t) may increase.

reaction_time(p,t,s3)=w31*(1−|target(s3)−prediction(p,t)|) [Equation 7]

(Here, target(s3) is s3/100, s3 is a fast reaction time preference setting value; w31 is a predetermined weight; prediction (p, t) is 1/rank (t); rank (t) is the location of ‘t’ in the list sorted in the order, in which the patient is predicted to have a fast reaction time, among the entire training content ‘T’).

For example, the value of rank (t) may increase as the patient is predicted to have a fast reaction time to the corresponding training content.

solution_time(p,t,s4)=w41*(1−|target(s4)−prediction(p,t)|) [Equation 8]

(Here, target(s4) is s4/100, s4 is a fast solution time preference setting value; w41 is a predetermined weight; prediction (p, t) is 1/rank (t); rank (t) is the location of ‘t’ in the list sorted in the order, in which the patient is predicted to have a fast solution time, among the entire training content ‘T’).

For example, the value of rank (t) may increase as the patient is predicted to have a fast solution time to the corresponding training content.

sensitivity(p,t,s5,[ts])=w51*ICF+w52*session_performance [Equation 9]

ICF=w511*(similarity([ts],t)/len[ts]) [Equation 10]

(Here, w511=s5/100; s5 is a sensitivity preference setting value; and, the similarity ([ts], t) is the number of times that the recommended training content ‘t’ matches ICF code in a training content list ([ts]) performed by the patient.)

session_performance=w521*(1−|new_target(s1,[ts])−correct_answer_rate(p,t,new_target(s1,[ts]),s2)|) [Equation 11]

(Here, w521 is s5/100; and new_target(s1,[ts]) is (s1−pre-training_correct_answer_rate ([ts]))+s1.)

For example, the recommendation model may calculate recommendation_score(p, t) of training content depending on set values of 0 to 100 and s1 to s5 and may generate a recommended content list including training content with a high recommendation score.

The training conducting unit 340 may calculate results for training content provided to a patient through a user terminal. Here, the training conducting unit 340 may calculate results for the training content, and the results for the training content include the number of problems solved by the patient, the time taken to be solved, the number of correct answers, and results for each area.

The result providing unit 350 may provide the results for training content. For example, while providing the result of performing the training content to the corresponding patient, the result providing unit 350 may compare the provided result with the previously performed training content to provide the corresponding result.

The profile updating unit 360 may update a patient profile based on the results of the training content. For example, when the patient's cognitive function is improved by performing the training content compared to the training content performed before the training content, the profile updating unit 360 may update the patient profile to reflect the improved cognitive function.

The patient cognitive score predicting unit 370 may generate a cognitive score prediction value by predicting a predefined cognitive score different from the patient's function-specific cognitive score based on a statistical value for each sub-function. For example, the patient cognitive score predicting unit 370 may predict and display MMSE, GDS, and CDR scores of the corresponding patient depending on the z-score value for each sub-function. That is, the patient cognitive score predicting unit 370 may provide convenience to the patient by comparing the patient with another patient having the same age, identifying the level of a cognitive function, and matching the identified result with another predefined cognitive score.

In the present disclosure, the patient cognitive score predicting unit 370 may calculate the accuracy of the cognitive score prediction value depending on the size of a parent population for calculating a statistical value for each sub-function. For example, the patient cognitive score predicting unit 370 may determine that the accuracy of matching with the cognitive score prediction value is high, as the size of the parent population for calculating a statistical value for each sub-function increases. That is, as the number of patients using the patient-customized training content recommendation device 130 increases, the patient cognitive score predicting unit 370 may collect more patient data to provide different cognitive score prediction values with higher accuracy. For example, the patient cognitive score predicting unit 370 may calculate the accuracy of the predicted cognitive score when data of the parent population exceeding a certain number of people is collected, and may display a simple prediction value and may not calculate the accuracy of the prediction value when the certain number of people is not exceeded.

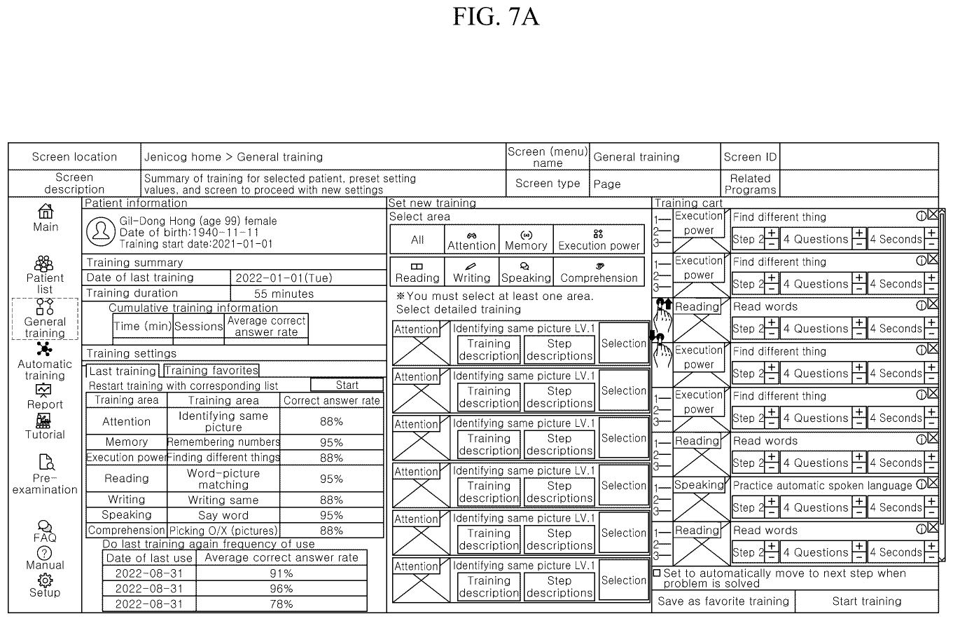

FIGS. 5 to 9 are diagrams illustrating screens in which the patient-customized training content recommendation device 130 according to an embodiment of the present disclosure is implemented.

Referring to FIG. 5, the patient-customized training content recommendation device 130 may identify a hospital name and a manager through a main screen, and may identify a registration date. Moreover, the patient-customized training content recommendation device 130 may count the number of therapists and the number of patients in real time through the main screen, may identify the amount used, may identify the number of recent patient registrations, and may identify necessary notices. Furthermore, through the main screen, the patient-customized training content recommendation device 130 may identify inquiries, may identify a registered therapist list, and may identify patient information.

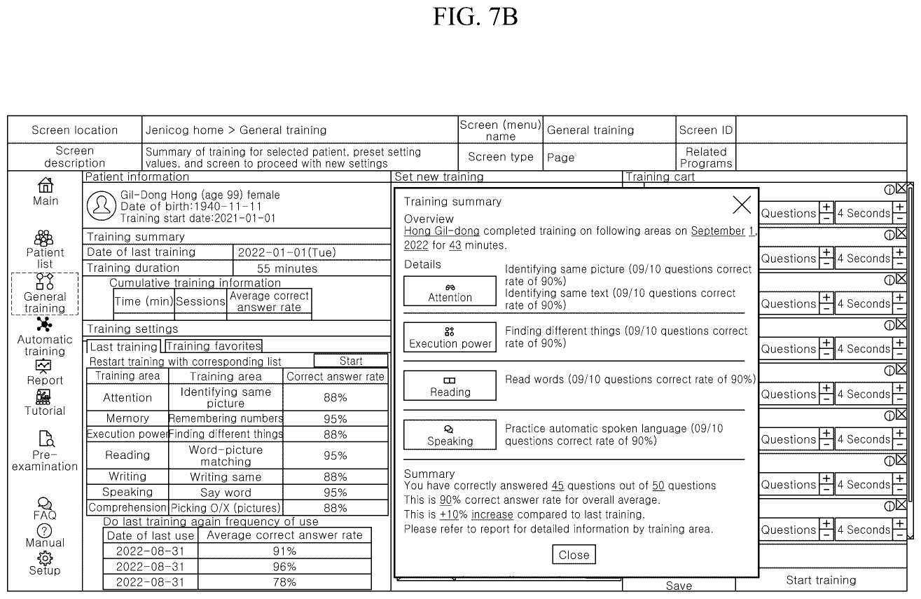

Referring to FIG. 6, the patient-customized training content recommendation device 130 may register/edit patients through a patient list screen, may write simple notes, and may identify a patient registration status. Also, the patient-customized training content recommendation device 130 may sort patients through the patient list screen and may identify detailed information of the selected patient.

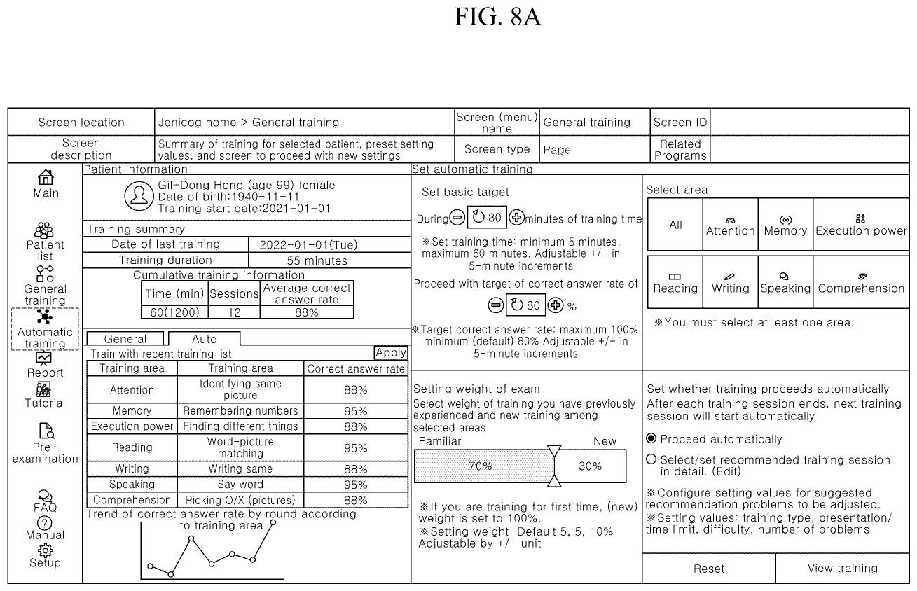

Referring to FIGS. 7A and 7B, the patient-customized training content recommendation device 130 may identify information of the selected patient and the training history of the patient through a general training page, may select the required area of training, and may set the order, difficulty, or the like for each training.

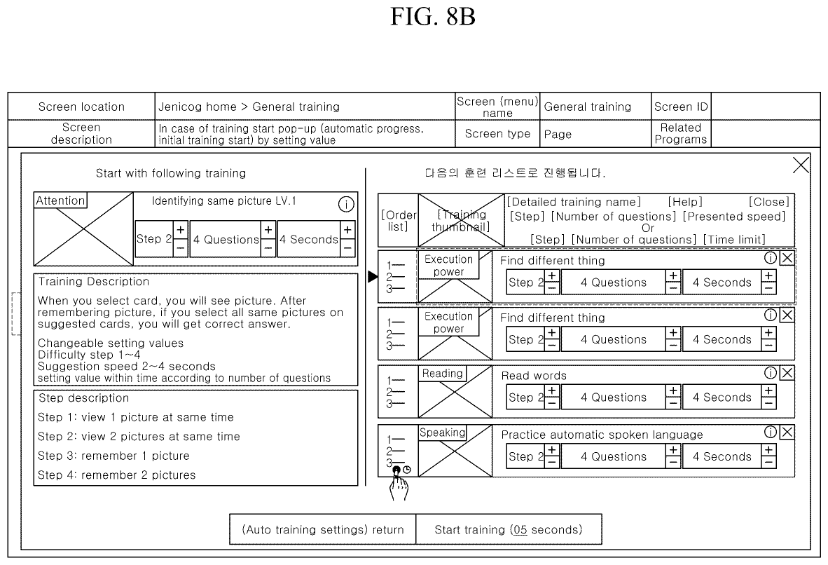

Referring to FIGS. 8A and 8B, the patient-customized training content recommendation device 130 may identify information of the selected patient through an automatic training page, may set a target time and a correct answer rate, may set a weight between previously experienced training and new training, may select an area that requires special treatment, and may select and control detailed setting for automatically recommended recommendation content.

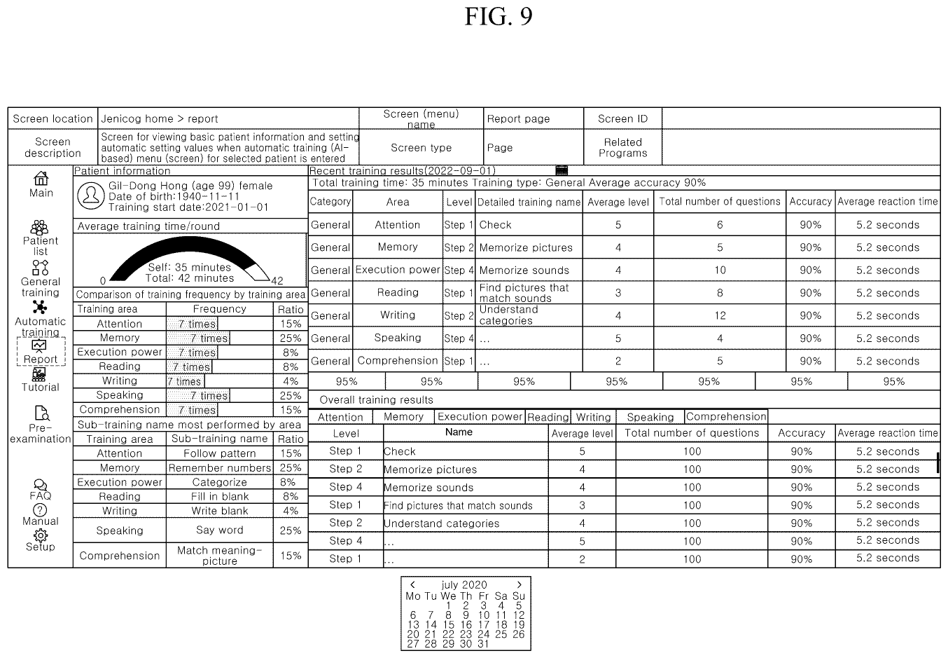

Referring to FIG. 9, the patient-customized training content recommendation device 130 may identify a training frequency for each detailed area through a report page, may identify information about the total training time, accuracy, and recent training, and may classify training content into detailed items to identify the insufficient part of a cognitive function.

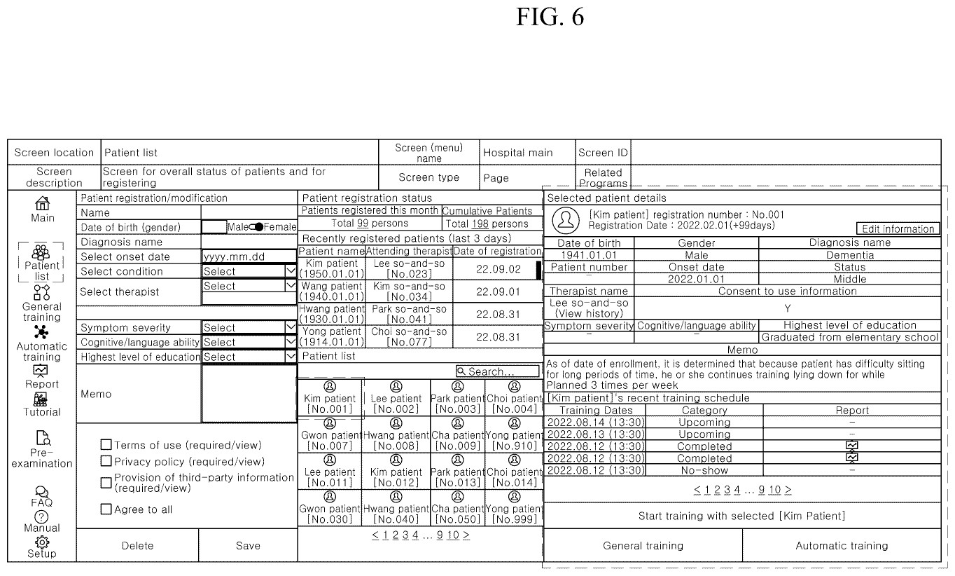

FIG. 4 is a diagram illustrating an order in which a patient-customized training content recommending method according to an embodiment of the present disclosure is performed.

Referring to FIG. 4, a patient-customized training content recommending method may receive a patient profile including basic information and cognitive information of a patient through the profile receiver 310 (S410).

The patient-customized training content recommending method may calculate the patient's function-specific cognitive score by evaluating the patient's cognitive ability based on the patient profile through the cognitive ability evaluating unit 320 (S420).

The patient-customized training content recommending method may recommend at least one training content through an artificial intelligence (AI) rehabilitation recommendation model based on a function-specific cognitive score through the training recommendation unit 330 (S430).

The patient-customized training content recommending method may calculate results for training content, which is provided to a patient through a user terminal, via the training conducting unit 340 (S440).

Meanwhile, the disclosed embodiments may be implemented in a form of a recording medium storing instructions executable by a computer. The instructions may be stored in a form of program codes, and, when executed by a processor, generate a program module to perform operations of the disclosed embodiments. The recording medium may be implemented as a computer-readable recording medium.

The computer-readable recording medium may include all kinds of recording media in which instructions capable of being decoded by a computer are stored. For example, there may be read only memory (ROM), random access memory (RAM), magnetic tape, magnetic disk, flash memory, optical data storage device, and the like.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

According to the above-mentioned problem solving means of the present disclosure, training content suitable for a patient is provided by identifying the patient's insufficient cognitive ability.

According to the above-mentioned problem solving means of the present disclosure, a predicted score is provided by predicting a score for a cognitive ability evaluation method different from a training method of obtaining the corresponding cognitive score by calculating a cognitive score.

Effects of the present disclosure are not limited to the effects mentioned above, and other effects not mentioned will be apparent by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A patient-customized training content recommendation device comprising:
a profile receiver configured to receive a patient profile including basic information and cognitive information of a patient;
a cognitive ability evaluating unit configured to calculate a function-specific cognitive score of the patient by evaluating a cognitive ability of the patient based on the patient profile;
a training recommendation unit configured to recommend at least one training content through an artificial intelligence (AI) rehabilitation recommendation model; and
a training conducting unit configured to calculate a result for the training content provided to the patient through a user terminal.

2. The patient-customized training content recommendation device of claim 1, wherein the basic information of the patient includes a current status, an age, a gender, and a highest level of education of the patient, and
wherein the cognitive information includes a result for the training content, and includes a separately pre-defined cognitive score when there is no training content already performed by the patient.

3. The patient-customized training content recommendation device of claim 2, wherein the cognitive ability evaluating unit is configured to:
define the function-specific cognitive score depending on a sub-function split stepwise; and
calculate a statistical value for the respective sub-function.

4. The patient-customized training content recommendation device of claim 3, wherein the cognitive ability evaluating unit is configured to define the function-specific cognitive score through:
a first step of defining at least one sub-function;
a second step of defining a detailed sub-function included in the at least one sub-function; and
a third step of performing labeling on the sub-function and the detailed sub-function to generate a label code for the sub-function and the detailed sub-function.

5. The patient-customized training content recommendation device of claim 4, wherein the cognitive ability evaluating unit is configured to:
calculate the function-specific cognitive score by summing the statistical value for each sub-function.

6. The patient-customized training content recommendation device of claim 3, wherein the AI rehabilitation recommendation model is configured to:
generate a recommended training content list including a specific label code depending on an input of the specific label code, and
wherein the training recommendation unit is configured to:
recommend the recommended training content list as the training content.

7. The patient-customized training content recommendation device of claim 1, wherein the AI rehabilitation recommendation model is configured to:
generate a recommended training content list based on a cluster, to which the patient belongs, by clustering at least one patient based on a performance result for specific training content.

8. The patient-customized training content recommendation device of claim 7, wherein the AI rehabilitation recommendation model is configured to:
generate, as the recommended training content list, training content performed by other patients in the cluster, to which the patient belongs, in addition to training content performed by the patient.

9. The patient-customized training content recommendation device of claim 7, wherein the AI rehabilitation recommendation model is configured to generate the recommended training content list through:
a first step of determining whether a correct answer rate of training content performed by the patient is greater than or equal to a first reference;
a second step of determining whether a reaction time for the training content performed by the patient is less than a second reference, when the correct answer rate of the patient is greater than or equal to the first reference; and
a third step of generating training content with increased difficulty as the recommended training content list when difficulty of the training content performed by the patient is not a maximum, when the reaction time of the patient is less than the second reference.

10. The patient-customized training content recommendation device of claim 7, wherein the AI rehabilitation recommendation model is configured to generate the recommended training content list through:
a first step of determining whether a correct answer rate of training content performed by the patient is less than a first reference;
a second step of determining whether the correct answer rate of the training content performed by the patient is greater than or equal to a third reference, when the correct answer rate of the patient is less than the first reference; and
a third step of generating training content having previous difficulty as the recommended training content list when the correct answer rate of the patient is greater than or equal to the third reference, and generating training content having reduced difficulty as the recommended training content list when the correct answer rate of the patient is less than the third reference.

11. The patient-customized training content recommendation device of claim 1, wherein the AI rehabilitation recommendation model includes:
a prediction model configured to predict a result of training content of the patient; and
a recommendation model configured to generate a recommended training content list based on a recommendation score calculated depending on a set target value.

12. The patient-customized training content recommendation device of claim 5, wherein the training content includes:
at least one cognitive training in which a training type, difficulty, a number of problems, and a solution time are set, and
wherein the training recommendation unit is configured to:
provide training content for reinforcing a sub-function of which the statistical value is low.

13. The patient-customized training content recommendation device of claim 12, wherein the training recommendation unit is configured to:
recommend the at least one training content by selecting cognitive training including most label codes of detailed sub-function included in the sub-function, of which the statistical value is low, through the AI rehabilitation recommendation model.

14. The patient-customized training content recommendation device of claim 3, further comprising:
a result providing unit configured to provide a result for the training content;
a profile updating unit configured to update the patient profile depending on a result of the training content; and
a patient cognitive score predicting unit configured to generate a cognitive score prediction value by predicting the predefined cognitive score different from the function-specific cognitive score of the patient depending on a statistical value for the respective sub-function,
wherein the patient cognitive score predicting unit is configured to:
calculate accuracy of the cognitive score prediction value depending on a size of a parent population for calculating a statistical value for the respective sub-function.

15. A patient-customized training content recommending method performed on a user terminal, the method comprising:
receiving a patient profile including basic information and cognitive information of a patient;
calculating a function-specific cognitive score of the patient by evaluating a cognitive ability of the patient based on the patient profile;
recommending at least one training content through an AI rehabilitation recommendation model; and
calculating a result for the training content provided to the patient through the user terminal.
